(19) United States
(12) Patent Application Publication
(10) Pub. No.: US 2008/0207670 A1
(43) Pub. Date: Aug. 28, 2008

(54) Dermally Applicable Formulations For Treating Skin Diseases in Animals

(75) Inventors: Kirkor Sirinyan, Bergisch Gladbach (DE); Holger Lehmann, Overland Park, KS (US)

Correspondence Address: BAYER HEALTHCARE LLC, P.O.BOX 390, SHAWNEE MISSION, KS, 66201, US

(73) Assignee: BAYER HEALTHCARE AG, Leverkusen (DE)

(21) Appl. No.: 11/575,474

(22) PCT Filed: Sep. 2, 2005
(86) PCT No.: PCT/EP05/09468

§ 371 Date: Sep. 21, 2007

Related U.S. Application Data
(60) Provisional application No. 60/610,493, filed on Sep. 16, 2004.

(51) Int. Cl. A61K 31/436; A61P 17/00

(52) U.S. Cl. 514/291

(57) ABSTRACT

The invention relates to pharmaceutical formulations comprising a tricyclic compound for the dermal treatment of skin diseases in animals.

The invention relates to pharmaceutical formulations comprising an immunosuppressive tricyclic lactone macrocycle for the dermal treatment of skin diseases in animals.

Immunosuppressive tricyclic lactone macrocycles, such as tacrolimus, which are suitable for treating skin diseases as well as other diseases are disclosed in EP-A 0 184 162 and also described in EP-A 444 659, EP-A-474 126 and WO 02/096419 in connection with particular pharmaceutical formulations. Other immunosuppressive tricyclic lactone macrocycles, such as pimecrolimus (cf. EP-A-427 680), sirolimus (also termed rapamycin, cf. U.S. Pat. No. 3,929,992) and ascrolimus (cf. WO 93/04680) are known in principle.

Formulations of these tricyclic macrocycles which are suitable for dermal treatment are likewise known in principle. For example, the application EP-A-0 315 978 describes tacrolimus-containing ethanolic preparations. According to application EP-A-0 474 126, it is possible to formulate these compounds in highly viscous ointment bases which contain an absorption accelerator, solvent and binder. In addition, applications WO 99/55332 and WO 94/28894 describe various highly viscous, pasty formulations which, in addition to the abovementioned ingredients, also comprise emulsifiers and/or thickeners. Application WO 02/096419 discloses highly viscous pastes which contain the tricyclic active compound, carbonates such as propylene carbonate, aliphatic ethers, such as diethylene glycol monoethyl ether, butylene glycol, long-chain fatty acid monoesters, such as isopropyl myristate, and diesters, such as diethyl adipate, and polymeric thickeners such as carboxyvinyl polymers.

Spot-on formulations of tacrolimus, or of other immunosuppressive tricyclic lactone macrocycles, for dermal use in animals have not yet been described. The known formulations for dermal use suffer from the disadvantage that they do not spread out on the animal coat, and therefore have to be rubbed in over large areas in some cases, leave behind ointment residues and lead to the animal coat becoming sticky at the application site and are consequently not suitable for treating animals conveniently and effectively.

The invention was therefore based on the object of providing a pharmaceutical which comprises immunosuppressive tricyclic lactone macrocycles, such as tacrolimus, and which permits simple and effective dermal application in animals.

This object is achieved by means of a spot-on formulation which comprises an immunosuppressive tricyclic lactone macrocycle.

Immunosuppressive tricyclic lactone macrocycles within the meaning of this application are, in particular:

compounds of the formula (I)

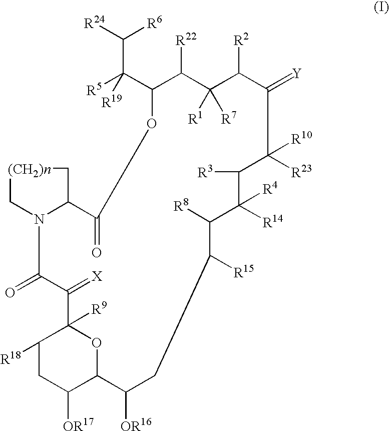

in which the vicinal pairs R¹ and R², R³ and R⁴ and R⁵ and R⁶ are, independently,
(a) 2 vicinal hydrogen atoms, where R² can also be an alkyl group,
(b) an additional bond between the carbon atoms to which they are bonded;
R⁷ is a hydrogen atom, a hydroxyl group, a protected hydroxyl group or an alkoxy group, or
R⁷ is, together with R¹, an oxo group;
R⁸ and R⁹ are, independently of each other, a hydrogen atom or a hydroxyl group;
R¹⁰ is a hydrogen atom, an alkyl group, an alkyl group which is substituted by one or more hydroxyl groups, an alkenyl group, an alkenyl group which is substituted by one or more hydroxyl groups, or an alkyl group which is substituted by an oxo group;
X is an oxo group, 2 hydrogen atoms, a hydrogen atom and a hydroxyl group, or the group of the formula —CH₂O—;
Y is an oxo group, a hydrogen atom and a hydroxyl group, 2 hydrogen atoms or a group of the formula N—NR¹¹R¹² or N—OR¹³;
R¹¹ and R¹² are, independently of each other, a hydrogen atom, an alkyl group, an aryl group or a tosyl group;
R¹³, R¹⁴, R¹⁵, R¹⁶, R¹⁷, R¹⁸, R¹⁹, R²² and R²³ are, independently of each other, a hydrogen atom or an alkyl group;
R²⁴ is an optionally substituted ring system which can contain one or more heteroatoms;
n is 1 or 2, and
Y, R¹⁰ and R²³, in addition to the above definitions, are, together with the carbon atoms to which they are bonded, a saturated or unsaturated 5- or 6-membered nitrogen-, sulphur- and/or oxygen-containing heterocyclic ring which is optionally substituted by one or more groups selected from alkyl, hydroxyl, alkoxy, benzyl, a group of the formula —CH₂Se(C₆H₅) and alkyl, which is substituted by one or more hydroxyl groups.

Within the meaning of this application, alkyl groups and the alkyl moiety of the alkoxy groups are, in particular, straight-chain or branched aliphatic hydrocarbon radicals having from 1 to 6 carbon atoms, for example methyl, ethyl, propyl, isopropyl, butyl, isobutyl, pentyl, neopentyl and hexyl.

Preferred examples of alkenyl groups are, in particular, straight-chain or branched aliphatic hydrocarbon radicals which contain from 1 to 6 carbon atoms and which possess at least one double bond, for example vinyl, propenyl (also termed allyl group), butenyl, methylpropenyl, pentenyl and hexenyl.

Preferred examples of aryl groups are phenyl, tolyl, xylyl, cumenyl, mesityl and naphthyl.

Preferred protecting groups for the hydroxyl and amino groups are: 1-(C₁₋₆-alkylthio)-C₁₋₆-alkyl groups, for example C₁₋₆-alkylthiomethyl groups such as methylthiomethyl, ethylthiomethyl, propylthiomethyl, isopropylthiomethyl, butylthiomethyl, isobutylthidmethyl, hexylthiomethyl, etc.

The methylthiomethyl group is very particularly preferred.

Mention may furthermore be made of trisubstituted silyl groups, such as tri-C₁₋₆-alkylsilyl (e.g. trimethylsilyl, triethylsilyl, tributylsilyl, tert-butyldimethylsilyl, tri-tert-butylsilyl, etc.) or C₁₋₆-alkyldiarylsilyl (e.g. methyldiphenylsilyl, ethyldiphenylsilyl, propyldiphenylsilyl, tert-butyl-diphenylsilyl, etc.). Preference is given to tri-C₁₋₄-alkylsilyl groups and C₁₋₄-alkyldiphenylsilyl groups, in particular the tert-butyldimethylsilyl group and the tert-butyldiphenylsilyl group.

Preference is furthermore given to acyl groups such as aliphatic, aromatic acyl groups, or an aliphatic acyl group which is substituted by an aromatic group, which are derived from carboxylic acids, sulphonic acids or carbamic acid.

Examples of aliphatic acyl groups are C₁₋₆-alkanoyl groups which, where appropriate, carry one or more substituents, such as the carboxyl radical, examples of such groups are formyl, acetyl, propionyl, butyryl, isobutyryl, valeryl, isovaleryl, pivaloyl, hexanoyl, carboxyacetyl, carboxy-propionyl, carboxybutyryl, carboxyhexanoyl, etc.; cyclo-C₁₋₆-alkoxy-C₁₋₆-alkanoyl groups which carry, where appropriate, one or more substituents such as C₁₋₆-alkyl, examples are cyclo-propyloxyacetyl, cyclobutyloxypropionyl, cycloheptyloxybutyryl, menthyloxyacetyl, menthyloxy-propionyl, menthyloxybutyryl, menthyloxypentanoyl, menthyloxyhexanoyl etc.; the camphorsulphonyl group or a C₁₋₆-alkylcarbamoyl group which carries one or more substituents such as carboxyl or protected carboxyl, examples are carboxy-C₁₋₆-alkylcarbamoyl (e.g. carboxymethyl-carbamoyl, carboxyethylcarbamoyl, carboxypropylcarbamoyl, carboxybutylcarbamoyl, carboxy-pentylcarbamoyl, carboxyhexylcarbamoyl, etc.), tri-C₁₋₆-alkylsilyl-C₁₋₆-alkoxycarbonyl-C₁₋₆-alkylcarbamoyl groups (e.g. trimethylsilylmethoxycarbonylethylcarbamoyl, trimethylsilylethoxy-carbonylpropylcarbamoyl, triethylsilylethoxycarbonylpropylcarbamoyl, tert-butyldimethylsilyl-ethoxycarbonylpropylcarbamoyl, trimethylsilylpropoxycarbonylbutylcarbamoyl, etc.).

Examples of aromatic acyl groups are, inter alia, aroyl groups which, where appropriate, carry one or more substituents such as nitro, examples are benzoyl, toluoyl, xyloyl, naphthoyl, nitrobenzoyl, dinitrobenzoyl, nitronaphthoyl, etc.; arenesulphonyl groups which, where appropriate, carry one or more substituents, such as halogen, examples are benzenesulphonyl, toluenesulphonyl, xylene-sulphonyl, naphthalenesulphonyl, fluorobenzenesulphonyl, chlorobenzenesulphonyl, bromo-benzenesulphonyl, iodobenzenesulphonyl, etc.;

Examples of aliphatic acyl groups which are substituted by an aromatic group are, inter alia, aryl-C₁₋₆-alkanoyl groups which, where appropriate, carry one or more substituents such as C₁₋₆-alkoxy or trihalogeno-C₁₋₆-alkyl. Examples are phenylacetyl, phenylpropionyl, phenyl-butyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl, etc.

Of the abovementioned acyl groups, particular preference is given to C₁₋₄-alkanoyl groups which, where appropriate, carry a carboxyl substituent, cyclo-C₅-C₆-alkoxy-C₁₋₄-alkanoyl groups which carry two C₁₋₄-alkyl substituents at the cycloalkyl unit, the camphorsulphonyl group, carboxy-C₁₋₄-alkylcarbamoyl groups, tri-C₁₋₄-alkylsilyl-C₁₋₁₄-alkoxycarbonyl-C₁₋₄-alkylcarbamoyl groups, the benzoyl group, which carries, where appropriate, one or two nitro groups, benzenesulphonyl groups which are optionally halogen-substituted, or the phenyl-C₁₋₄-alkanoyl group which is optionally substituted by a C₁₋₄-alkoxy substituent, and the trihalogeno-C₁₋₄-alkyl group. Very particular preference is given to acetyl, carboxypropionyl, menthyloxyacetyl, camphorsulphonyl, benzoyl, nitrobenzoyl, dinitrobenzoyl, iodobenzenesulphonyl and 2-trifluoromethyl-2-methoxy-2-phenylacetyl.

Preferred examples of the “5- or 6-membered nitrogen-, sulphur- and/or oxygen-containing heterocyclic ring” comprise the pyrrolyl group and the tetrahydrofuryl group.
R²⁴ is an optionally substituted ring system which can contain one or more heteroatoms. R²⁴ is preferably the cyclo-C₅₋₇-alkyl group which carries suitable substituents, where appropriate. Preferred examples are given below:
   (a) the 3,4-dioxocyclohexyl group
   (b) a 3-R²⁰-4-R²¹-cyclohexyl group in which
      R²⁰ is hydroxyl, alkoxy, an oxo group or the group —OCH₂OCH₂CH₂OCH₃, and
      R²¹ is hydroxyl, —OCN, an alkoxy group, optionally substituted heteroaryloxy, 1- or 2-tetrazolyl, the group —OCH₂OCH₂CH₂OCH₃, a protected hydroxyl group, chlorine, bromine, iodine, aminooxalyloxy, an azide group, p-tolyloxythiocarbonyloxy or R²⁵R²⁶CHCOO— in which
         R²⁵ is optionally protected hydroxyl or protected amino, and
         R²⁶ is hydrogen or methyl, or
         R²⁰ and R²¹ together form an oxygen atom in an epoxide ring, or
   (c) a cyclopentyl group which is substituted by methoxymethyl, optionally protected hydroxymethyl, acyloxymethyl (in which the acyl unit contains an optionally quaternized dimethylamino group or an optionally esterified carboxyl group), by one or more optionally protected amino and/or hydroxyl groups, or by aminooxalyloxymethyl. A preferred example is the 2-formylcyclopentyl group.

The optionally substituted heteroaryl unit of the optionally substituted heteroaryloxy can be one of those which are specified in R¹ of the compound from EP-A-532,088, with this document being hereby expressly incorporated herein by reference; 1-hydroxyethylindol-5-yl is preferred.

The compounds of formula (I), and their pharmaceutically acceptable salts, are in principle disclosed in WO 02/096419 and the documents which are cited therein.

A representative example of the compounds of formula (I) is tacrolimus (SK 506) of the following formula

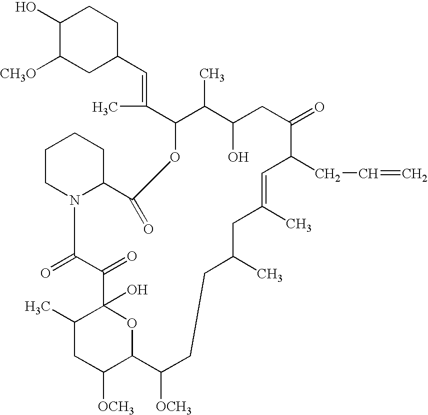

### Chemical Name:

17-allyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo-[22.3.1.0^{4,9}]octacos-18-ene-2,3,10,16-tetraone

Preferred compounds of formula (I) are those in which the vicinal pairs
R³ and R⁴ or R⁵ and R⁶ are, independently of each other, an additional bond between the carbon atoms to which they are bonded;
R⁸ and R²³ are, independently, a hydrogen atom;
R⁹ is a hydroxyl group;

R¹⁰ is a methyl group, an ethyl group, a propyl group or an allyl group;
X is 2 hydrogen atoms or an oxo group;
Y is an oxo group;
R¹⁴, R¹⁵, R¹⁶, R¹⁷, R¹⁸, R¹⁹ and R²² are in each case a methyl group,
R²⁴ is a 3-R²⁰-4-R²¹-cyclohexyl group in which
   R²⁰ is hydroxyl, an alkoxy group, an oxo group or the group —OCH₂OCH₂CH₂OCH₃, and
   R²¹ is hydroxyl, —OCN, an alkoxy group, optionally substituted heteroaryloxy, 1- or 2-tetrazolyl, the group —OCH₂OCH₂CH₂OCH₃, a protected hydroxyl group, chlorine, bromine, iodine, aminooxalyloxy, an azido group, paratolyloxythiocarbonyloxy or R²⁵-R²⁶—CHCOO—, in which
      R²⁵ is optionally protected hydroxyl or protected amino, and
      R²⁶ is hydrogen or methyl, or
   R²⁰ and R²¹ together form an oxygen atom in an epoxide ring and
   n is an integer 1 or 2.

Other particularly preferred compounds of the formula (I) apart from tacrolimus are ascomycin compounds, such as halogenated ascomycins, e.g. 33-epichloro-33-deoxyascomycin, termed pimecrolimus, which is disclosed as Example 66a in EP-A 427 680.

Pimecrolimus has the following formula

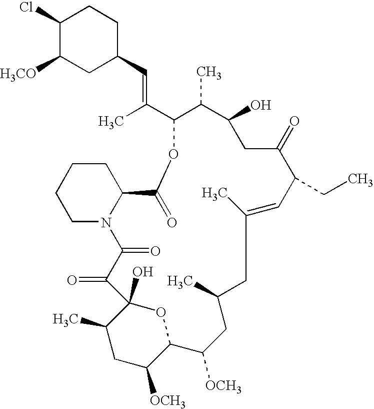

Other particularly preferred immunosuppressive tricyclic lactone macrocycles are the compound sirolimus (also termed rapamycin, see U.S. Pat. No. 3,929,992) having the formula

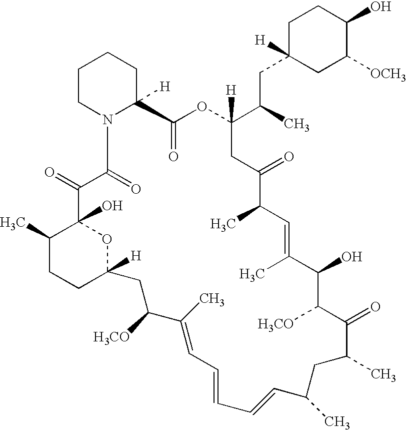

and the compound ascrolimus (WO 93/04680) having the formula

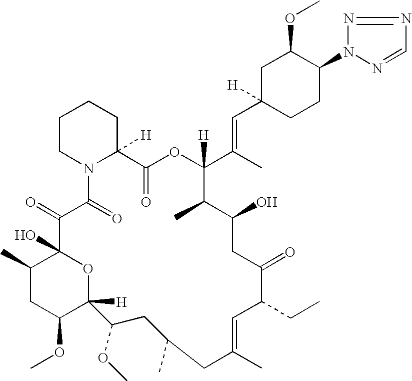

The immunosuppressive tricyclic lactone macrocycles can also be used in the form of their non-toxic pharmaceutically acceptable salts. Suitable salts are salts with organic and inorganic bases, in particular alkali metal salts, such as sodium salts and potassium salts, alkaline earth metal salts, such as calcium salts and magnesium salts, and ammonium salts, such as the triethylammonium salt and the N-benzyl-N-methylamine salt.

The abovementioned immunosuppressive tricyclic lactone macrocycles can be present in the form of different conformers and stereoisomers, such as optical and geometric isomers based on asymmetric carbon atoms or double bonds. These conformers and isomers can also be used within the context of the present invention. The immunosuppressive tricyclic lactone macrocycles can furthermore also be present in the form of their solvates, such as hydrates or ethanolates. These latter can also be used within the context of the present invention.

Where appropriate, the pharmaceuticals according to the invention can also comprise further suitable pharmaceutical active compounds in addition to the immunosuppressive tricyclic lactone macrocycles.

Formulations which are suitable for dermal application in animals should, in particular, be homogeneous and of low viscosity and should spread well. In addition to the spontaneous spreading behaviour, such formulations should also exhibit very good activity, toleration by the target animal, low toxicity for homeotherms and exceptional long-term stability, e.g. in the single-dose plastic tubes which are customary for spot-on formulations and which have a capacity of from 0.5 to 6.0 ml.

The average viscosity of the formulations according to the invention is from 3 to 12 mPa·s, preferably from 4 to 7.5 mPa·s, particularly preferably from 4.5 to 6.5 mPa·s.

In principle, all the customary spot-on solvents are suitable for preparing the novel spot-on formulations. Customary spot-on solvents which may be mentioned, in particular, are: aliphatic and aromatic alcohols, such as isopropanol, ethanol, methanol, octanol and benzyl alcohol; organic carbonates, such as propylene carbonate or ethylene carbonate, pyrrolidones, such as N-methylpyrrolidone, 2-pyrrolidone or octylpyrrolidone, aliphatic ethers, in particular glycol ethers, such as diethylene glycol monomethyl ether and dipropylene glycol monomethyl ether, esters, for example isopropyl myristate, and ketals, such as solketal. Naturally, the said solvents can be provided with the customary stabilizers, UV absorbers, acidifying agents and oligomeric as well as polymeric spreading agents.

The invention furthermore relates, in particular, to a pharmaceutical comprising
(a) from 0.01 to 10% by weight (based on the total mass) of an immunosuppressive tricyclic lactone macrocycle,
(b) from 5 to 32% by weight (based on the total mass) of a solvent selected from: cyclic carbonates, benzyl alcohol and dimethyl sulphoxide (DMSO) or mixtures of these solvents,
(c) from 50 to 75% by weight (based on the total mass) of glycol ethers,
(d) from 0.01 to 0.75% by weight (based on the total mass) of phenolic antioxidants,
(e) from 0 to 7.5% by weight (based on the total mass) of triglycerides or esters of dihydric alcohols,
(f) from 0 to 7.5% by weight of moisture-attracting, moisture-binding short-chain glycols.

The formulations according to the invention comprise from 0.01 to 10% by weight, preferably from 0.1 to 5% by weight, particularly preferably from 0.2 to 3.0% by weight, of immunosuppressive tricyclic active compound. Where appropriate, the formulations according to the invention can also comprise further active compounds.

The solvent cited under (b) is present in the pharmaceuticals according to the invention in proportions of from 5 to 32% by weight, preferably of from 7.5 to 25% by weight, particularly preferably from 15 to 25% by weight.

According to one embodiment, the solvents concerned are cyclic carbonates, in particular propylene carbonate or ethylene carbonate.

According to another embodiment, the solvent employed is benzyl alcohol.

According to another embodiment, the solvent employed is dimethyl sulphoxide.

According to another embodiment, mixtures of the abovementioned solvents, for example ethylene carbonate/benzyl alcohol or propylene carbonate/benzyl alcohol, can also be employed as solvent (b).

In the case of solvent mixtures, the mixing ratio is customary in the range of from 90:10 to 10:90, preferably of from 80:20 to 20:80.

The aliphatic ethers cited under (c) are usually glycol monoalkyl ethers or glycol dialkyl ethers. The glycol moiety can be derived from ethylene glycol or from propylene glycol; the alkyl radicals usually contain from 1 to 4 carbon atoms. Preferred examples which may be mentioned are: diethylene glycol monoethyl ether and dipropylene glycol monomethyl ether. The aliphatic ethers are as a rule present in the pharmaceuticals in quantities of from 50 to 75% by weight, preferably of at least 60% by weight, particularly preferably of at least 67.5% by weight.

Phenolic antioxidants are preferably BHA (butylhydroxyanisole) and/or BHT (butylhydroxytoluene). The quantities of BHT and/or BHA which are preferred in the pharmaceuticals according to the invention are from 0.01 to 0.75% by weight, particularly preferably from 0.05 to 0.25% by weight, very particularly preferably about 0.1%.

The triglycerides or esters of dihydric alcohols which are cited under (e) are in principle known as pharmaceutical auxiliary substances. They contain, as the alcohol component, a dihydric or trihydric alcohol containing up to three carbon atoms, for example ethylene glycol, propylene glycol or, in the case of the triglycerides, glycerol. The acid component of the ester comprises fatty acids which contain from 6 to 18 carbon atoms and which may be straight-chain or branched and also be monounsaturated or polyunsaturated. As a rule, medium-chain fatty acids containing from 6 to 12 carbon atoms are preferred. It is possible to use mixed esters or else mixtures of different ester types. Examples of suitable triglycerides are caprylic/capric triglycerides or caprylic/capric/linoleic triglycerides.

Esters of propylene glycol with caprylic acid and/or capric acid (propylene glycol octanoate decanoate) are similarly preferred. These esters can be obtained from Sasol Germany GmbH/Witten under the trade names Miglyol 840 (propylene glycol octanoate decanoate, CAS No. 68583-51-7) and Miglyol 812 (caprylic/capric triglycerides, CAS No. 73398-61-5). It is also possible to use their polyethylene oxide-modified, polypropylene oxide-modified and/or propylene carbonate-modified derivatives.

Low-viscosity esters having a viscosity (20° C.) of from 8 to 40 mPa·s, particularly preferably of from 26 to 35 mPa·s, are preferably employed in the pharmaceuticals according to the invention.

The esters cited under (e) are present in the pharmaceuticals according to the invention in quantities of from 0 to 10% by weight, preferably from 2.5 to 7.5% by weight, particularly preferably from 2.5 to 5% by weight.

In a number of cases, it is advisable to add moisture-attracting, moisture-binding short-chain glycols, in quantities of 0-7.5% by weight, preferably of from 2.5 to 5% by weight, to the formulations according to the invention. Ethylene glycol and propylene glycol may be taken as examples of these glycols.

In addition, absorption accelerators, i.e. what are termed penetration enhancers, such as terpenes, oleic acid, etc., may be admixed with the formulations according to the invention. Absorption accelerators are known per se. Examples of suitable absorption accelerators are given, for example, on pages 199-230 in the textbook R. C. Scott, R. H. Guy and J. Hadgraft Prediction of Percutaneous Penetration Methods, Measurements, Modelling Associate Editor SM Tittensor (1989), which is hereby expressly incorporated herein by reference.

In principle, the said novel formulations are suitable for treating all animals such as domestic animals and productive animals. They are preferably used for mammals, particularly for domestic animals such as cats and dogs. A use for horses is also preferred.

The volume of the novel formulations which is preferably applied is from 0.025 to 0.25 ml/kg, particularly preferably from 0.05 to 0.1 ml/kg, of the body weight of the animal to be treated.

The said formulations are quite outstandingly suitable for treating skin diseases, in particular those skin diseases which have a primary or secondary immunological genesis, such as atopic dermatitis, contact dermatitis, inflammatory skin diseases, otitis externa, otitis media, pemphigus, lupus and perianal fistulas.

Exploratory experiments using compositions which did not contain any active compound (what are termed “placebo preparations”) indicated outstanding spontaneous spreading behaviour on animal coats, very good tolerance by the target animal, low toxicity in homeotherms, ready applicability and very good long-term stability, in all climate zones, in the single-dose polypropylene tubes which are used for spot-on application. Suitable polypropylene tubes usually have capacities of from 0.5 to 6.0 ml and a wall thickness of from 300 to 500 μm.

It is expected that it will be possible, without difficulty, to aliquot the formulations according to the invention into single-dose plastic tubes on conventional non-explosion-proof spot-on filling devices.

### EXAMPLES

The following examples propose recipes for preparing formulations according to the invention. In principle, these formulations can be prepared by stirring the active compound tacrolimus in the given ingredients at RT (room temperature).

### Example 1

100.0 ml of homogeneous formulation consisting of

| | |
|---|---|
| 0.5 g | of tacrolimus |
| 20.0 g | of propylene carbonate |
| 76.84 g | of diethylene glycol monoethyl ether |
| 5.4 g | of caprylic/capric triglyceride (viscosity range (20° C.) |
| | of from 28 to 32 mPa · s and having the trade name Miglyol |
| | 812, from Sasol Germany GmbH, D-58453 Witten |
| 0.1 g | of BHT (butylhydroxytoluene) |

### Example 2

100.0 ml of homogeneous formulation consisting of

| | |
|---|---|
| 0.5 g | of tacrolimus |
| 20.0 g | of benzyl alcohol |
| 74.48 g | of diethylene glycol monoethyl ether |
| 5.4 g | of caprylic/capric triglyceride (viscosity range (20° C.) |
| | of from 28 to 32 mPa · s and having the trade name Miglyol |
| | 812, from Sasol Germany GmbH, D-58453 Witten |
| 0.1 g | of BHT |

### Example 3

100.0 ml of homogeneous formulation consisting of

| | |
|---|---|
| 0.5 g | of tacrolimus |
| 20.0 g | of propylene carbonate |
| 74.48 g | of dipropylene glycol monomethyl ether |
| 5.4 g | of caprylic/capric triglyceride (viscosity range (20° C.) |
| | of from 28 to 32 mPa · s and having the trade name Miglyol |
| | 812, from Sasol Germany GmbH, D-58453 Witten |
| 0.1 g | of BHA |

### Example 4

100.0 ml of homogeneous formulation consisting of

| | |
|---|---|
| 0.5 ml | of tacrolimus |
| 20.0 ml | of propylene carbonate/benzyl alcohol (1:1) |
| 75.62 ml | of diethylene glycol monoethyl ether |
| 5.4 ml | of caprylic/capric triglyceride (viscosity range (20° C.) |
| | of from 28 to 32 Pa · s, molar mass approx. 520, and |
| | having the trade name Miglyol 812, from Sasol Germany |
| | GmbH, D-58453 Witten |
| 0.1 ml | of BHA (butylhydroxyanisole) |

### Example 5

100.0 ml of homogeneous formulation consisting of

| | | |
|---|---|---|
| 0.5 | ml | of tacrolimus |
| 20.0 | g | of propylene carbonate |
| 74.64 | g | of diethylene glycol monoethyl ether |
| 7.4 | g | of caprylic/capric triglyceride (viscosity range (20° C.) |
| | | of from 27 to 30 mPa · s, molar mass approx. 520, and |
| | | having the trade name Miglyol 810, from Sasol Germany |
| | | GmbH, D-58453 Witten |
| 0.1 | g | of BHA |

### Example 6

100.0 ml of homogeneous formulation consisting of

| | |
|---|---|
| 0.5 g | of tacrolimus |
| 20.0 g | of benzyl alcohol |
| 72.64 g | of diethylene glycol monoethyl ether |
| 7.4 g | of caprylic/capric triglyceride (viscosity range (20° C.) |
| | of from 27 to 30 mPa · s, molar mass approx. 520, and |
| | having the trade name Miglyol 810, from Sasol Germany |
| | GmbH, D-58453 Witten |
| 0.1 g | of BHA |

### Example 7

100.0 ml of homogeneous formulation consisting of

| | |
|---|---|
| 1.5 g | of tacrolimus |
| 19.0 g | of ethylene carbonate |
| 77.68 g | of diethylene glycol monoethyl ether |
| 5.4 g | of caprylic/capric triglyceride (viscosity range (20° C.) of |
| | from 28 to 32 mPa · s and having the trade name Miglyol 812, |
| | from Sasol Germany GmbH, D-58453 Witten |
| 0.1 g | of BHT |

### Example 8

100.0 ml of homogeneous formulation consisting of

| | |
|---|---|
| 1.5 g | of tacrolimus |
| 19.0 g | of propylene carbonate |
| 69.64 g | of diethylene glycol monoethyl ether |
| 5.4 g | of caprylic/capric triglyceride (viscosity range (20° C.) |
| | of from 28 to 32 mPa · s and having the trade name Miglyol |
| | 812, from Sasol Germany GmbH, D-58453 Witten |
| 5.0 g | of propylene glycol |
| 0.1 g | of BHT |

1. Pharmaceutical for spot-on application, comprising an immunosuppressive tricyclic lactone macrocycle.

2. Pharmaceutical comprising
(a) from 0.01 to 10% by weight (based on the total mass) of an immunosuppressive tricyclic lactone macrocycle,
(b) from 5 to 32% by weight (based on the total mass) of a solvent selected from: cyclic carbonates, benzyl alcohol and dimethyl sulphoxide (DMSO) or mixtures of these solvents,
(c) from 50 to 75% by weight (based on the total mass) of aliphatic ethers,
(d) from 0.01 to 0.75% by weight (based on the total mass) of phenolic antioxidants,
(e) from 0 to 7.5% by weight (based on the total mass) of triglycerides or esters of dihydric alcohols,
(f) from 0 to 7.5% by weight of moisture-attracting, moisture-binding short-chain glycols.

3. Pharmaceutical according to claim 1, comprising an immunosuppressive tricyclic lactone macrocycle selected from: tacrolimus, pimecrolimus, sirolimus and ascrolimus.

4. Pharmaceutical according to claim 3, comprising tacrolimus.

5. Pharmaceutical according to one of the preceding claims comprising ethylene carbonate.

6. Pharmaceutical according to one of the preceding claims comprising propylene carbonate.

7. Pharmaceutical according to one of the preceding claims, comprising diethylene glycol monoethyl ether.

8. Pharmaceutical according to one of the preceding claims, comprising dipropylene glycol monomethyl ether.

9. Use of a pharmaceutical according to one of the preceding claims for controlling skin diseases in animals.

10. Use of an immunosuppressive tricyclic lactone macrocycle for producing a spot-on pharmaceutical for controlling skin diseases in animals.
